(19) United States
(12) Patent Application Publication
(10) Pub. No.: US 2022/0370037 A1
(43) Pub. Date: Nov. 24, 2022

(54) VASCULAR TISSUE CHARACTERIZATION DEVICES, SYSTEMS, AND METHODS

(71) Applicants: KONINKLIJKE PHILIPS N.V., Eindhoven (NL); PHILIPS IMAGE GUIDED THERAPY CORPORATION, SAN DIEGO, CA (US)

(72) Inventors: Yang SUN, RANCHO CORDOVA, CA (US); Yiqun YANG, SACRAMENTO, CA (US); Anuja NAIR, HIGHLAND HEIGHTS, OH (US); Andrew HANCOCK, SACRAMENTO, CA (US)

(21) Appl. No.: 17/764,058

(22) PCT Filed: Sep. 24, 2020
(86) PCT No.: PCT/EP2020/076641

§ 371 Date: Mar. 25, 2022

Related U.S. Application Data
(60) Provisional application No. 62/913,224, filed on Oct. 10, 2019.

(51) Int. Cl. A61B 8/12; A61B 8/14; A61B 8/08

(52) U.S. Cl. CPC A61B 8/12; A61B 8/14; A61B 8/5246; A61B 8/5207; A61B 8/4488

(57) ABSTRACT

Ultrasound image devices, systems, and methods are provided. An ultrasound imaging system, comprising an intraluminal imaging device comprising a flexible elongate member configured to be positioned within a patient's body lumen and an ultrasound transducer array coupled to the flexible elongate member, the ultrasound transducer array configured to transmit ultrasound energy into the body lumen and to receive ultrasound echoes associated with the body lumen; and a processor circuit in communication with the intraluminal imaging device and configured to receive, from the ultrasound transducer array, first signal data corresponding to the received ultrasound echoes; transform the first signal data based on a first parameter associated with a reference tissue characterization data; characterize the transformed first signal data based on the reference tissue characterization data; generate a first image of the body lumen based on the characterization; and output, to a display in communication with the processor circuit, the first image.

### TECHNICAL FIELD

The present disclosure relates generally to intraluminal imaging devices, such an intravascular ultrasound (IVUS) imaging catheters, and in particular, to identifying vascular tissue components based on reference vascular tissue characterization data and providing virtual histology (VH) images based on identified vascular tissue components.

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. An IVUS device including one or more ultrasound transducers is passed into the vessel and guided to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed. IVUS imaging can provide detailed and accurate measurements of lumen and vessel sizes, plaque areas and volumes, and location of key anatomical landmarks. IVUS imaging allows physicians to evaluate the size and type of a lesion, select a treatment device (e.g., a stent) based on the evaluated lesion size, and subsequently evaluate the treatment success.

There are two types of IVUS catheters commonly in use today: rotational and solid-state. For a typical rotational IVUS catheter, a single ultrasound transducer element is located at the tip of a flexible driveshaft that spins inside a plastic sheath inserted into the vessel of interest. The transducer element is oriented such that the ultrasound beam propagates generally perpendicular to the axis of the device. As the driveshaft rotates, the transducer is periodically excited with a high voltage pulse to emit a short burst of ultrasound. The same transducer then listens for the returning echoes reflected from various tissue structures. The IVUS imaging system assembles a two-dimensional display of the vessel cross-section from a sequence of pulse/acquisition cycles occurring during a single revolution of the transducer.

Solid-state IVUS catheters carry an ultrasound imaging assembly that includes an array of ultrasound transducers distributed around its circumference along with one or more integrated circuit controller chips mounted adjacent to the transducer array. The solid-state IVUS catheters are also referred to as phased array IVUS transducers or phased array IVUS devices. The controllers select individual transducer elements (or groups of elements) for transmitting an ultrasound pulse and for receiving the ultrasound echo signal. By stepping through a sequence of transmit-receive pairs, the solid-state IVUS system can synthesize the effect of a mechanically scanned ultrasound transducer but without moving parts (hence the solid-state designation).

Ultrasound echoes or backscatter collected from the IVUS imaging devices during an ultrasound examination can be processed and analyzed using various signal processing and/or imaging algorithms to determine clinical features and/or conditions associated with the patient under the ultrasound examination. For example, the radio frequency (RF) signal from the backscatter data can be correlated with known histology data to provide classification of the patient's vasculature. The classification of the vasculature from the RF backscatter data may be referred to as virtual histology (VH). The classification may include identifying boundary features within the vasculature and plaque and determining the composition of each patient's atherosclerotic plaques from the RF backscatter data.

In an example, RF backscatter signal collected from an IVUS imaging device can be transformed into a frequency domain. Power spectral characteristics of the RF backscatter data can be analyzed to provide tissue characterization and/or classification. For example, frequency parameters can be identified from the RF backscatter data and correlated with signal properties of known tissue types, such as fibrous, fibro-fatty, dense calcium and necrotic core. The identified frequency parameters and the association with corresponding tissue types can be stored in a database. The database can be reviewed and validated by physicians and/or experts. The database can be adjusted based on the review and validation. The review, the validation, and/or the adjustments can be repeated in multiple iterations. After the database is validated to provide accurate tissue characterization, the database may be applied to IVUS imaging data acquired during a clinical examination, for example, in real time, to determine vascular tissue types. VH images can be generated based on the identified vascular tissue types to assist physicians in diagnosing and/or assessing patient's conditions.

The signal properties or parameters used for vascular tissue characterization can be dependent on the signal path of the IVUS system. However, as advanced hardware components, software components, signal processing algorithms, and/or image processing algorithms are introduced into the IVUS system, the signal properties of the RF backscatter data can vary. Accordingly, the tissue characterization database may not be suitable for some advanced IVUS systems and/or may not provide tissue characterization with high accuracy when used with the advanced IVUS systems. While a new tissue characterization database can be generated after each hardware and/or software upgrade is applied to the IVUS system, the effort in collecting new data, correlating with histology samples, and/or validating the database can be significant and time-consuming (e.g., years) and thus may not be practical.

### SUMMARY

While existing IVUS imaging system have proved useful, there remains a need for improved systems and techniques for providing accurate and consistent tissue characterization with system upgrades. Embodiments of the present disclosure provide techniques for a first intraluminal imaging system to reuse a well-validated tissue characterization database (e.g., an ex-vivo vessel sample database) generated from a second intraluminal imaging system for tissue characterization. For example, the second system may be a current or previous generation system, and the first system may be a new or next generation system. The first system and the second system may employ the same type of intraluminal imaging device, but may include different receive signal paths (e.g., different receiver frontend circuitries, filters, and/or gains). In the disclosed embodiments, the first system may acquire radio frequency (RF) backscatter data (e.g., ultrasound echo responses) from an imaging examination and condition the raw RF backscatter data by a transfer function (e.g., a finite impulse response (FIR) bandpass filter). The transfer function is configured such that the conditioned RF backscatter data may have the same or similar signal characteristics as raw RF backscatter data acquired from the second system. In an embodiment, the tissue characterization database may store frequency parameters identified from RF backscatter data in association with corresponding tissue components. In an embodiment, after conditioning the raw RF backscatter data, the first system may apply the same or substantially similar signal processing techniques and/or the signal processing blocks as the second system to the conditioned RF backscatter data for tissue characterization. The signal processing may include baseband (BB) conversion, BB beamforming, RF remodulation, and/or frequency analysis. In an embodiment, the first system may generate a tissue composition map (e.g., virtual histology (VH) image) for a vessel object under imaging and overlay the tissue composition map on top of a corresponding brightness-mode (B-mode) image of the vessel object.

In one embodiment, an ultrasound imaging system includes an intraluminal imaging device comprising a flexible elongate member configured to be positioned within a body lumen of a patient and an ultrasound transducer array coupled to the flexible elongate member, the ultrasound transducer array configured to transmit ultrasound energy into the body lumen and to receive ultrasound echoes associated with the body lumen; and a processor circuit in communication with the intraluminal imaging device and configured to receive, from the ultrasound transducer array, first signal data corresponding to the received ultrasound echoes; transform the first signal data to second signal data based on a first parameter associated with a reference tissue characterization data; characterize the second signal data based on the reference tissue characterization data; generate a first image of the body lumen based on the characterization; and output, to a display in communication with the processor circuit, the first image.

In some embodiments, wherein the first signal data includes radio frequency (RF) backscatter data corresponding to the received ultrasound echoes. In some embodiments, wherein the first parameter includes a frequency parameter associated with the reference tissue characterization data. In some embodiments, wherein the first parameter includes a power spectral parameter associated with the reference tissue characterization data. In some embodiments, wherein the processor circuit configured to transform the first signal data to the second signal data includes applying a filter to the first signal data, the filter determined based on the first parameter. In some embodiments, wherein the processor circuit configured to transform the first signal data to the second signal data includes applying the filter to a portion of the first signal data based on a field of view (FOV) parameter associated with the reference tissue characterization data. In some embodiments, wherein the processor circuit is further configured to convert the second signal data to baseband signal data; perform beamforming on the baseband signal data to produce focused data; and convert the focused data to radio frequency (RF) signal data, and wherein the processor circuit configured to characterize the second signal data is further configured to characterize the RF signal data based on the reference tissue characterization data. In some embodiments, wherein the processor circuit is further configured to adjust at least one of a time parameter or a gain parameter of the first signal data based on a second parameter associated with the reference tissue characterization data. In some embodiments, wherein the processor circuit is further configured to generate a brightness-mode (B-mode) image of the body lumen based on the first signal data; and output, to the display, a combined image including the B-mode image and the first image, wherein the first image includes a tissue composition map of the body lumen. In some embodiments, wherein the reference tissue characterization data is associated with at least one of a calcified tissue type, a fibrous tissue type, a fibro-lipidic tissue type, or a calcified necrosis tissue type. In some embodiments, wherein the processor circuit includes a field programmable gate array (FPGA) configured to transform the first signal data to the second signal data based on the first parameter associated with the reference tissue characterization data. In some embodiments, the system further comprises a memory configured to store program code for transforming the first signal data to the second signal data based on the first parameter associated with the reference tissue characterization data, wherein the processor circuit includes a processor in communication with the memory, and wherein the processor circuit configured to transform the first signal data to the second signal data includes executing the program code by the processor.

In one embodiment, a method of ultrasound imaging, comprising receiving, at a processor circuit in communication with an intraluminal imaging device including an ultrasound transducer array, first signal data corresponding to ultrasound echoes associated with a body lumen; transforming the first signal data to second signal data based on a first parameter associated with a reference tissue characterization data; characterizing the second signal data based on the reference tissue characterization data; generating a first image of the body lumen based on the characterization; and outputting, to a display in communication with the processor circuit, the first image.

In some embodiments, wherein the first signal data includes radio frequency (RF) backscatter data corresponding to the ultrasound echoes. In some embodiments, wherein the first parameter includes a frequency parameter associated with the reference tissue characterization data. In some embodiments, wherein the transforming includes applying a filter to the first signal data, the filter determined based on the first parameter. In some embodiments, wherein the transforming includes applying the filter to a portion of the first signal data based on a field of view (FOV) parameter associated with the reference tissue characterization data. In some embodiments, the method further comprises converting the second signal data to baseband signal data; performing beamforming on the baseband signal data to produce focused data; and converting the focused data to radio frequency (RF) signal data, and wherein the characterizing includes characterizing the RF signal data based on the reference tissue characterization data. In some embodiments, the method further comprises adjusting at least one of a time parameter or a gain parameter of the first signal data based on a second parameter associated with the reference tissue characterization data. In some embodiments, the method further comprises generating a B-mode image of the body lumen based on the first signal data, wherein the outputting includes outputting, to the display, a combined image including the B-mode image and the first image, wherein the first image includes a tissue composition map of the body lumen.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an intraluminal ultrasound imaging system, according to aspects of the present disclosure.

FIG. 2 is a schematic diagram illustrating a reference tissue characterization data generation system, according to aspects of the present disclosure.

FIG. 3 illustrates a tissue characterization scheme, according to aspects of the present disclosure.

FIG. 4 is a schematic diagram illustrating an intraluminal ultrasound imaging system that provides tissue characterization, according to aspects of the present disclosure.

FIG. 5 illustrates example brightness mode (B-mode) images and virtual histology (VH) images generated from an intraluminal ultrasound imaging system, according to aspects of the present disclosure.

FIG. 6 is a schematic diagram of a processor circuit, according to embodiments of the present disclosure.

FIG. 7 is a flow diagram of a tissue characterization method, according to aspects of the disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

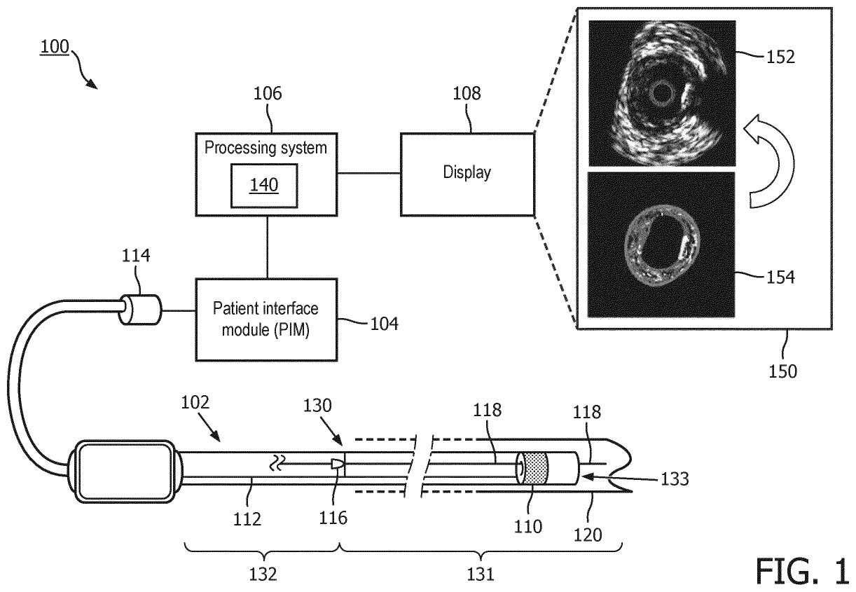

FIG. 1 is a schematic diagram of an intraluminal ultrasound imaging system 100, according to aspects of the present disclosure. The system 100 may include an intraluminal imaging device 102, a patient interface module (PIM) 104, a processing system 106, and a display 108. The intraluminal imaging device 102 may be a catheter, a guide wire, or a guide catheter. The intraluminal imaging device 102 can be referred to as an interventional device and/or a diagnostic device. In some instances, the intraluminal imaging device 102 can be a therapeutic device. The processing system 106 may include various computing hardware and/or software components. In some instances, the processing system 106 may be a console, a computer, a laptop, a tablet, or a mobile device. The display 108 may be a monitor. In some embodiments, the display 108 may be an integrated component of the processing system 106.

The intraluminal imaging device 102 may include a flexible elongate member sized and shaped for insertion into the vasculature of a patient. The flexible elongate member may include a distal portion 131 and a proximal portion 132. The intraluminal imaging device 102 may include an imaging component 110 mounted at the distal portion 131 near a distal end 133 of the intraluminal imaging device 102. The intraluminal imaging device 102 may be inserted into a body lumen or vessel 120 of the patient. For example, the intraluminal imaging device 102 can be inserted into a patient's vessel 120 to capture images of the structure of the vessel 120, measure the diameter and/or length of the vessel 120 to guide stent selection, and/or measure blood flow in the vessel 120. The vessel 120 may be any artery or vein within a vascular system of a patient, including cardiac vasculature, peripheral vasculature, neural vasculature, renal vasculature, and/or any other suitable anatomy/lumen inside the body. In some embodiments, the vessel 120 may be a venous vessel, a pulmonary vessel, a coronary vessel, or a peripheral vessel. For example, the device 102 may be used to examine any number of anatomical locations and tissue types, including without limitation, organs including the liver, heart, kidneys, gall bladder, pancreas, lungs, esophagus; ducts; intestines; nervous system structures including the brain, dural sac, spinal cord and peripheral nerves; the urinary tract; as well as valves within vasculature or the heart, chambers or other parts of the heart, and/or other systems of the body. In addition to natural structures, the device 102 may be used to examine man-made structures such as, but without limitation, heart valves, stents, shunts, filters and other devices.

In an embodiment, the imaging component 110 may include ultrasound transducers or acoustic elements configured to emit ultrasonic energy towards the vessel 120. The emission of the ultrasonic energy may be in the form of pulses. The ultrasonic energy is reflected by tissue structures and/or blood flows in the vessel 120 surrounding the imaging component 110. The reflected ultrasound echo signals or echo responses are received by the ultrasound transducers in the imaging component 110. In some instances, the imaging component 110 may be configured for brightness-mode (B-mode) imaging to capture images of vessel structures or to measure vessel diameters and lengths for stent selection. In some other instances, the imaging component 110 may be configured for Doppler color flow imaging to provide blood flow measurements. In yet some other instances, the imaging component 110 may be configured to operate in a dual-mode to provide both B-mode imaging data and Doppler flow measurements.

In some embodiments, the ultrasound transducers in the imaging component 110 are phased-array transducers, which may be configured to emit ultrasound energy at a frequency of about 10 megahertz (MHz), about 20 MHz, about 30 MHz, or about 40 MHz. The imaging component 110 can include an array of acoustic elements with any number of acoustic elements (e.g., about 16, 32, 64, or 128) arranged in any suitable configuration. For example, the acoustic elements can be circumferentially arranged around a longitudinal axis of the device 102. In some other embodiments, the imaging component 110 may be alternatively configured to include a rotational transducer to provide similar functionalities. For example, the device 102 can include a rotating drive cable coupled to a housing in which the imaging component 110 is positioned. The imaging component 110 can be a single transducer/acoustic element. Rotation of the drive cable causes corresponding rotation of the imaging component 110.

An ultrasound transducer array of ultrasound imaging device includes an array of acoustic elements configured to emit ultrasound energy and receive echoes corresponding to the emitted ultrasound energy. In some instances, the array may include any number of ultrasound transducer elements. For example, the array can include between 2 acoustic elements and 10000 acoustic elements, including values such as 2 acoustic elements, 4 acoustic elements, acoustic elements, 64 acoustic elements, 128 acoustic elements, 500 acoustic elements, 812 acoustic elements, 3000 acoustic elements, 9000 acoustic elements, and/or other values both larger and smaller. In some instances, the transducer elements of the array may be arranged in any suitable configuration, such as a linear array, a planar array, a curved array, a curvilinear array, a circumferential array, an annular array, a phased array, a matrix array, a one-dimensional (1D) array, a 1.x dimensional array (e.g., a 1.5D array), or a two-dimensional (2D) array. The array of transducer elements (e.g., one or more rows, one or more columns, and/or one or more orientations) can be uniformly or independently controlled and activated. The array can be configured to obtain one-dimensional, two-dimensional, and/or three-dimensional images of patient anatomy.

In some embodiments, the imaging component 110 may include a piezoelectric micromachined ultrasound transducer (PMUT), capacitive micromachined ultrasonic transducer (CMUT), single crystal, lead zirconate titanate (PZT), PZT composite, piezoelectric/piezoresistive, other suitable transducer types, and/or combinations thereof. The ultrasound transducer elements of the array are in communication with (e.g., electrically coupled to) electronic circuitry. For example, the electronic circuitry can include one or more transducer control logic dies. The electronic circuitry can include one or more integrated circuits (IC), such as application specific integrated circuits (ASICs). In some embodiments, one or more of the ICs can include a microbeamformer (μBF). In other embodiments, one or more of the ICs includes a multiplexer circuit (MUX).

The PIM 104 facilitates communication of signals between the processing system 106 and the intraluminal imaging device 102 to control the operation of the imaging component 110. The PIM 104 can include hardware and/or software components configured to generate control signals for configuring the imaging component 110, triggering transmitter circuits to cause the imaging component 110 to emit ultrasound waves, and transfer echo signals captured by the imaging component 110 to the processing system 106. With regard to the echo signals, the PIM 104 forwards the received signals and, in some embodiments, performs preliminary signal processing prior to transmitting the signals to the processing system 106. In examples of such embodiments, the PIM 104 performs amplification, filtering, and/or aggregating of the data. In an embodiment, the PIM 104 also supplies high- and low-voltage direct current (DC) power to support operation of the circuitry within the imaging component 110.

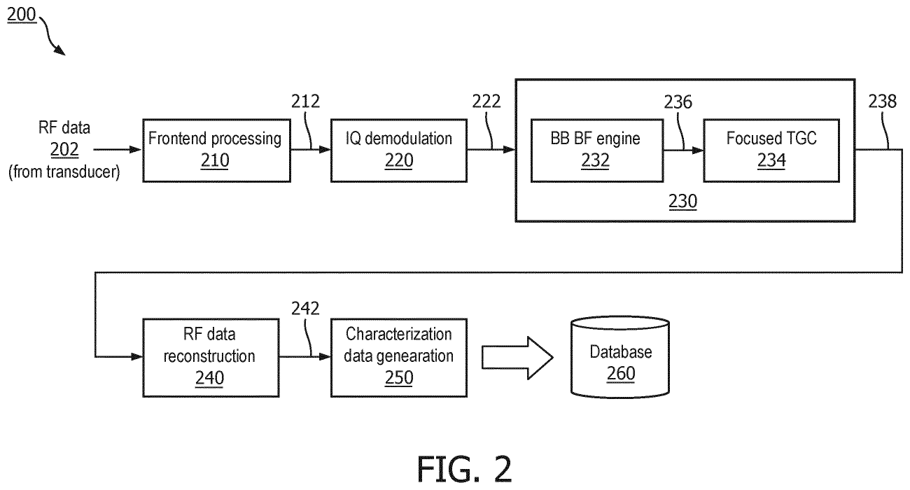

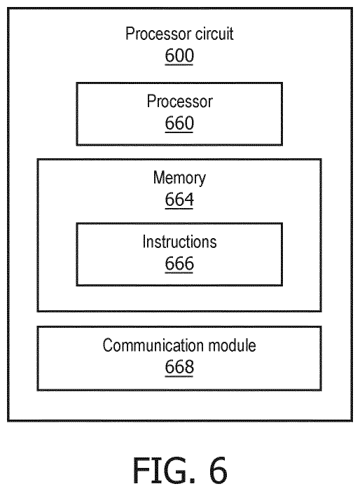

The processing system 106 receives the echo data from the imaging component 110 and/or transmits controls to the imaging component 110 by way of the PIM 104. The processing system 106 can include one or more processors and a memory as described in greater detail herein (shown in FIG. 6). The processing system 106 can be operable to facilitate the features of the system 100 described herein, for example, aspects of FIGS. 2-5 and 7. In an embodiment, the processing system 106 is configured to process the echo response to reconstruct an image of the tissue structures in the vessel 120 surrounding imaging component 110. For example, for B-mode imaging, the strengths or the amplitudes of the echo responses may be converted to brightness or intensity levels for gray-scale image display. The processing system 106 outputs image data such that an image (e.g., the image 152) of the vessel 120, such as a cross-sectional image of the vessel 120, is displayed on the display 108.

In some embodiments, the intraluminal imaging device 102 includes some features similar to traditional solid-state IVUS catheters, such as the EagleEye® Platinum, Eagle Eye® Platinum ST, Eagle Eye® Gold, and Visions® PV catheters available from Volcano Corporation and those disclosed in U.S. Pat. No. 7,846,101 hereby incorporated by reference in its entirety. For example, the intraluminal imaging device 102 further includes an electrical cable 112 extending along the longitudinal body of the intraluminal imaging device 102. The cable 112 is a transmission line bundle including a plurality of conductors, including one, two, three, four, five, six, seven, or more conductors. It is understood that any suitable gauge wire can be used for the conductors. In an embodiment, the cable 112 can include a four-conductor transmission line arrangement with, e.g., 41 American wire gauge (AWG) wires. In an embodiment, the cable 112 can include a seven-conductor transmission line arrangement utilizing, e.g., 44 AWG wires. In some embodiments, 43 AWG wires can be used. In some other embodiments, the intraluminal imaging device 102 includes some features similar to traditional rotational IVUS catheters, such as the Revolution® catheter available from Volcano Corporation and those disclosed in U.S. Pat. Nos. 5,601,082 and 6,381,350, each of which is hereby incorporated by reference in its entirety. In some embodiments, the intraluminal imaging device 102 includes components or features similar or identical to those disclosed in U.S. Pat. Nos. 4,917,097, 5,368,037, 5,453,575, 5,603,327, 5,779,644, 5,857,974, 5,876,344, 5,921,931, 5,938,615, 6,049,958, 6,0854,109, 6,123,673, 6,165,128, 6,283,920, 6,309,339; 6,033,357, 6,457,365, 6,712,767, 6,725,081, 6,767,327, 6,776,763, 6,779,257, 6,7854,157, 6,899,682, 6,962,567, 6,976,965, 7,097,620, 7,226,417, 7,641,4854, 7,676,910, 7,711,413, and 7,736,317, each of which is hereby incorporated by reference in its entirety.

The cable 112 terminates in a PIM connector 114 at a proximal end of the intraluminal imaging device 102. The PIM connector 114 electrically couples the cable 112 to the PIM 104 and physically couples the intraluminal imaging device 102 to the PIM 104. In an embodiment, the intraluminal imaging device 102 further includes a guide wire exit port 116 disposed near a junction 130 at which the distal portion 131 is coupled to the proximal portion 132. Accordingly, in some instances the intraluminal imaging device 102 is a rapid-exchange catheter. The guide wire exit port 116 allows a guide wire 118 to be inserted towards the distal end 133 in order to direct the intraluminal imaging device 102 through the vessel 120.

In an embodiment, the system 100 can be used in various stages of tissue characterization. For example, the processing system 106 further includes a tissue characterization component 140. In an embodiment, the tissue characterization component 140 is configured to receive and store characterization data (e.g., tissue type data, etc.) in a database (e.g., a database 260 in FIG. 2). In this regard, after a vascular object (e.g., the vessel 120) has been interrogated, where ultrasound energy is emitted to the vessel 120 and ultrasound echoes are collected, the vascular object is cross-sectioned for histology. The cross section is then prepared with a fixing and staining process that is well known in the art. The staining process allows a clinician to identify a tissue type(s). The identified tissue type (e.g., characterization data) is then provided to the tissue characterization component 140 and stored in the database. The characterization component 140 is also configured to perform spectral analysis on the RF signals of the ultrasound echoes (e.g., backscatter data) to identify spectral properties and/or spectral parameters. The characterization component 140 can store the identified spectral properties and/or parameters in association with corresponding identified tissue types in the database. The database can be reviewed and validated by experts and/or physicians and may be tuned based on the reviews to provide accurate tissue characterization and/or classification.

In an embodiment, during a clinical examination, the tissue characterization component 140 is configured to receive ultrasound echo data obtained from the interrogation of a vascular object (e.g., the vessel 120), determine spectral properties or parameters associated with the tissue types and/or characteristics for the received ultrasound echo data, and utilize the spectral parameters stored in the database (e.g., histology data) to identify one or more tissue types and/or characteristics associated with the ultrasound echo data.

In an embodiment, the tissue characterization component 140 is configured to create a VH image 154 of the interrogated vascular object and identify at least one corresponding region on a corresponding B-mode image 152 of the vascular object. The VH image 154 may include representation of tissue types determined from the characterization. In some examples, the representation can be in the form of a color-coded tissue composition map. The characterization component 140 may identify a region of interest (ROI) on the VH image 154 and identify a corresponding region on the B-mode image 152 using various algorithms including warping and/or morphing the VH image 154 to substantially fit the contour of the B-mode image 152. The tissue characterization component 140 can provide a combined image 150 including the B-mode image 152 and the VH image 154 to the display 108. For simplicity of illustration, FIG. 1 illustrates the B-mode image 152 and the VH image 154 as separate images. However, the VH image 154 may be displayed as an overlay on top of the B-mode image 152 as indicated by the block arrow.

In an embodiment, the system 100 may be used to generate a reference tissue characterization database and may subsequently be upgraded to include advanced and/or improved hardware, software, and/or algorithms implemented on the PIM 104 and/or the system 106. According to embodiments of the present disclosure, the system 100 can be configured to reuse the same reference tissue characterization database and/or tissue characterization component after the upgrade to provide tissue characterization. Mechanisms for acquiring RF backscatter data, generating a reference tissue characterization database, and/or utilizing a reference tissue characterization database for tissue characterization with any upgrades in the signal path of the system 100 are described in greater detail herein.

FIG. 2 is a schematic diagram illustrating a reference tissue characterization data generation system 200, according to aspects of the present disclosure. The system 200 is substantially similar to the system 100 and provides a more detailed view of a receive signal path associated with tissue characterization data generation. The system 200 includes a frontend processing component 210, an in-phase, quadrature-phase (IQ) demodulation component 220, a focusing engine 230, an RF demodulation component 240, a characterization data generation component 250, and a tissue characterization database 260. The frontend processing component 210, the IQ demodulation component 220, the focusing engine 230, the RF data reconstruction component 240, and the characterization data generation component 250 can be implemented using any suitable combination of hardware and software components. The frontend processing component 210, the IQ demodulation component 220, the focusing engine 230, the RF data reconstruction component 240, and the characterization data generation component 250 may be implemented in the PIM 104, the processing system 106, and/or any intermediate processing system between the PIM 104 and the processing system 106.

The frontend processing component 210 is configured to receive raw RF data 202 from an ultrasound transducer (e.g., the imaging component 110). For example, the ultrasound transducer may be coupled to a catheter (e.g., the intraluminal imaging device 102) and inserted into a vessel object (e.g., the vessel 120) under ultrasound examination. The RF data 202 may include per-channel ultrasound echoes or backscatter data received from each acoustic element of the transducer. The frontend processing component 210 may include amplifiers, filters, and/or gain controls configured to condition the received RF data 202. In some embodiments, the frontend processing component 210 can further include analog-to-digital converters (ADCs) configured to sample the analog RF data 202 to provide digital RF data.

The IQ demodulation component 220 is coupled to the frontend processing component 210 and configured to convert the RF data 212 to a baseband (BB) to provide BB IQ data 222. The RF data 212 may include real-valued data samples. The BB IQ data 222 may include complexed IQ pairs. The IQ demodulation component 220 may perform down-conversion, low-pass filtering, and/or decimation. The down-conversion converts the RF data 212 from the RF to baseband, for example, by down-mixing the RF data 212 with two sinusoidal signals with a 90 degrees phase difference. The low-pass filtering removes negative frequency components and noise outside the desired frequency bandwidth. The decimation reduces the sampling rate of the BB IQ data 222. The low-pass filtering and the decimation preserve the information content of the RF data 212 in the BB IQ data 222. In some embodiments, the IQ demodulation component 220 is an analog IQ demodulator. In such embodiments, the frontend processing component 210 may not include ADCs and the RF data 212 includes analog RF signal data. In some other embodiments, the IQ demodulation component 220 is a digital IQ demodulator. In such embodiments, the frontend processing component 210 may include the ADCs and the RF data 212 includes digitized RF signal data.

The focusing engine 230 is coupled to the IQ demodulation component 220. The focusing engine may include a BB beamform (BF) engine 232 coupled to a focused time-gain-compensation (TGC) component 234. The BF engine 232 may include delay elements and summing elements configured to apply appropriate time-delays to at least a subset of the BB IQ data 222 and combine the time-delayed data to form beamformed data 236. The beamformed data may include a plurality of focused scan lines. The focused TGC component 234 can include weighting elements configured to adjust the amplitudes of the samples along the scan lines in the BB IQ data 222 to account for gain variations in the axial direction (e.g., as a function of depth) due to ultrasound signal attenuation.

The RF data reconstruction component 240 is coupled to the focusing engine 230 and configured to reconstruct RF data from the post-focused data 238. The reconstruction of RF data from the complexed IQ data 222 is the reversal of the complex demodulation performed by the IQ demodulation component 220. Thus, the RF data reconstruction may also be referred to as RF remodulation. The RF data reconstruction component 240 can apply interpolation, filtering, and up-conversion or up-mixing on the post-focused data 238 to provide focused RF data 242 suitable for tissue component analysis. As an example, the received RF data 202 may be centered at about 20 MHz, the BB IQ data 222 may be centered at about 0 Hz, and the reconstructed RF data 242 may be re-modulated to be centered at about 20 MHz.

The characterization data generation component 250 is coupled to the RF data reconstruction component 240. The characterization data generation component 250 can be substantially similar to the tissue characterization component 140 of FIG. 1. The characterization data generation component 250 is configured to determine spectral parameters associated special properties of the focused RF data 242 for tissue characterization. The characterization data generation component 250 is configured to store the spectral parameters in association with tissue composition information of the vessel object in the database 260. The database 260 may be referred to as a VH database. The tissue classification information may indicate the types of plaque components that may be present in the vessel object. Some examples of plaque components may include necrotic core, dense calcium, fibrous, fibro-fatty, collagen, fibrolipid, calcium, and/or calcified necrosis. The system 200 may repeat the process of RF backscatter data (e.g., the RF data 202) collection, spectral analysis, and tissue composition association for tissue samples of various tissue types until a sufficient amount of histology data is collected for the database 260. The database 260 can be reviewed and validated by experts and physicians. The database 260 can be tuned and adjusted based on the reviews and feedbacks received from the experts and physicians. After the database 260 is validated, the database 260 can be applied during a clinical examination for tissue characterization. Mechanisms for generating the characterization data and using the data for tissue classification are described in greater detail herein.

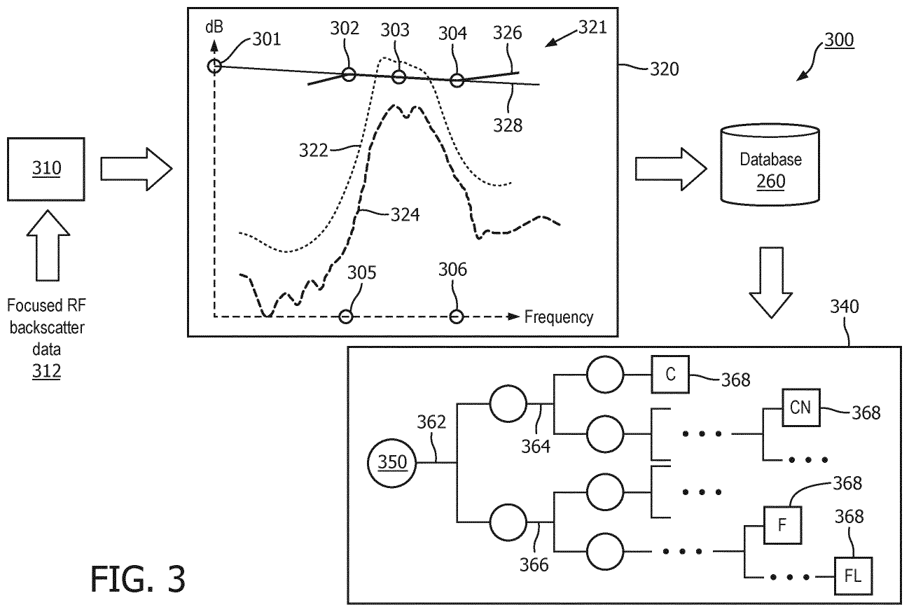

FIG. 3 illustrates a tissue characterization scheme 300, according to aspects of the present disclosure. The scheme 300 can be employed by the systems 100 and/or 200. In particular, the scheme 300 can be implemented by the tissue characterization component 140 of FIG. 1 and/or the characterization data generation component 250. The scheme 300 receives focused RF backscatter data 312, for example, corresponding to the RF data 242 provided by the system 200. The RF data 312 is time-domain signal data including a plurality of scan lines representing a vessel object (e.g., the vessel 120) under ultrasound examination. The scheme 300 determines an ROI 310 from the RF data 312. The scheme 300 may use any suitable border detection (e.g., automatic border detection) techniques for determining the ROI 310. The scheme 300 converts the time-domain RF data 312 to a frequency domain. Different types and densities of tissue may absorb and reflect the ultrasound waves differently. Thus, the RF data 312 includes characteristics of the types of tissue in the vessel object under the imaging. Accordingly, differences in the RF data 312 along each scan line can be determined by performing frequency analysis 320 on the RF data 312. The frequency analysis 320 can use any suitable techniques including spectral analysis, autoregressive (AR) modelling, wavelet decomposition, and/or curvelet decomposition.

As an example, a frequency plot 321 for the frequency analysis 320 is shown in FIG. 3, where the x-axis represents frequency in units of MHz and the y-axis represents power in units of decibels (dB). The frequency analysis 320 can include computing a frequency spectrum 322 (e.g., a power spectrum) for each scan line in the ROI 310. The frequency analysis 320 can include computing an average frequency spectrum 324 by averaging the frequency spectrums over the width of the ROI 310. The frequency analysis 320 can include computing a normalized spectrum 326 by subtracting the average frequency spectrum 324 from the frequency spectrum 322. The frequency analysis 320 can include determining a linear regression line 328 from the normalized spectrum 326. The frequency analysis 320 can include identifying spectral properties (e.g., frequency parameters) from the normalized spectrum 326 and the linear regression line 328 for tissue component identification. Some example frequency parameters may include a y-intercept parameter 301, a maximum power parameter 302, a frequency location 305 corresponding to the maximum power, a mid-band parameter 303, a minimum power parameter 304, a frequency location 306 corresponding to the minimum power, a slope of the linear regression line 328, and/or integrated backscatter data (e.g., an integral between two points on the spectrum curve 326). In an example, the integrated backscatter data may refer to the definite integral between the maximum power parameter 302 and the minimum power parameter 304. In other words, the integrated backscatter may be the area under the normalized spectrum curve 326 between frequency 305 and frequency 306.

In some embodiments, the frequency analysis 320 can include analyzing each scan line by segments (e.g., corresponding to certain imaging depths). For example, the frequency analysis 320 can determine frequency parameters similar to the parameters 301-306, the slope, and/or the integrated backscatter data for each segment of a scan line and for each scan line in the ROI 310. The scheme 300 may repeat the frequency analysis 320 for multiple vessel object samples to collect signal properties (e.g., frequency parameters) for tissue characterization.

After collecting a sufficient amount of signal property information for tissue characterization, the scheme 300 stores the identified frequency parameters in association with tissue types corresponding to the vessel object samples in the database 260. The database 260 may be implemented in a variety of ways including a data file, an array, a table, a linked list, a tree structure, a database, neural network, combinations of these and multiple components of each if desired.

In an embodiment, the scheme 300 applies statistical classification tree techniques to store the frequency parameters and associated tissue types in the database 260. For example, the scheme 300 may store predetermined signal properties (e.g., the frequency parameters) and corresponding tissue types in the database 260 as a classification tree or a regression tree having branch node conditions based on the predetermined tissue signal properties and one or more leaf nodes that identify a tissue component.

An example of a statistical classification tree 340 is shown in FIG. 3. The tree 340 includes a root node 350 that branches based on the signal properties compiled from a statistical algorithm, and may also include other secondary parameters (e.g., patient body-mass index, patient conditions, patient demographics, and/or imaging parameters associated with the generation and/or emission of ultrasound waves). For example, a first branch 362 in a first branch level is based on a value of mid-band fit (e.g., the mid-band parameter 303), a second branch 364 in a second branch level is based on a value of minimum power (e.g., the minimum power parameter 304), and a third branch 366 in the second branch level is based on a value of slope (e.g., the slope of the linear regression line 328). The branching condition at the first branch 362 may be a mid-band fit threshold. The branching condition at the second branch 364 may be a minimum power threshold. The branching condition at the third branch 366 may be a slope threshold. In general, the tree 340 may include any suitable number of branch levels and any suitable number of branches similar to the branches 362, 364, and 366. Each branch (e.g., the branches 362, 364, and 366) in the tree 340 may include a branching condition that is based on a frequency parameter, such as the y-intercept parameter 301, the maximum power parameter 302, the maximum power frequency location 305, the mid-band parameter 303, the minimum power parameter 304, the minimum power frequency location 306, the slope of the linear regression line 328, and/or the integrated backscatter data described above.

The tree 340 terminates at leaf nodes 368 (shown as rectangular boxes) that represent a particular type of tissue. In this example, the leaf nodes 368 indicate a calcified tissue type (represented by “C”), a fibrous tissue type (represented by the “F”), a fibro-lipidic tissue type (represented by the “FL”) and a calcified necrosis tissue type (represented by “CN”). In some instances, the tree 340 may include multiple leaf nodes 368 having the same tissue type. In general, the leaf nodes 368 can indicate any suitable type of vascular components, such as normal tissue, lumen, types of plaque components that may be present. In some other embodiment, spectral parameters associated with mass lesion component may be collected and analyzed and correlated with corresponding mass lesion samples. In such embodiments, the leaf nodes 368 can indicate a type of mass lesion component, such as normal tissue, cancerous tissue, hyperplastic tissue, hypertrophic tissue, immune cells, and types of tumor cells.

The database 260 and/or the classification tree 340 can be used for real-time tissue classification during an ultrasound examination in a clinical setting. For example, by inputting a set of signal properties collected from an ultrasound examination, the classification tree 340 can be traversed in accordance with the branching conditions and lead to a leaf node 368 that identifies the type of tissue matching the inputted signal properties. Mechanisms for performing tissue characterization are described in greater detail herein.

In some embodiments, the scheme 300 may employ mechanisms similar or identical to those disclosed in U.S. Pat. No. 7,074,188 B2, U.S. Patent Application Publication No. 2014/0163369 A1, and “Coronary Plaque Classification With Intravascular Ultrasound Radiofrequency Data Analysis” by Anuja Nair, Barry D. Kuban, E. Murat Tuzcu, Paul Schoenhagen, Steven E. Nissen, D. Geoffrey Vince, Circulation. 2002; 106: 2200-2206, each of which is hereby incorporated by reference in its entirety, for tissue characterization data generation and/or tissue characterization.

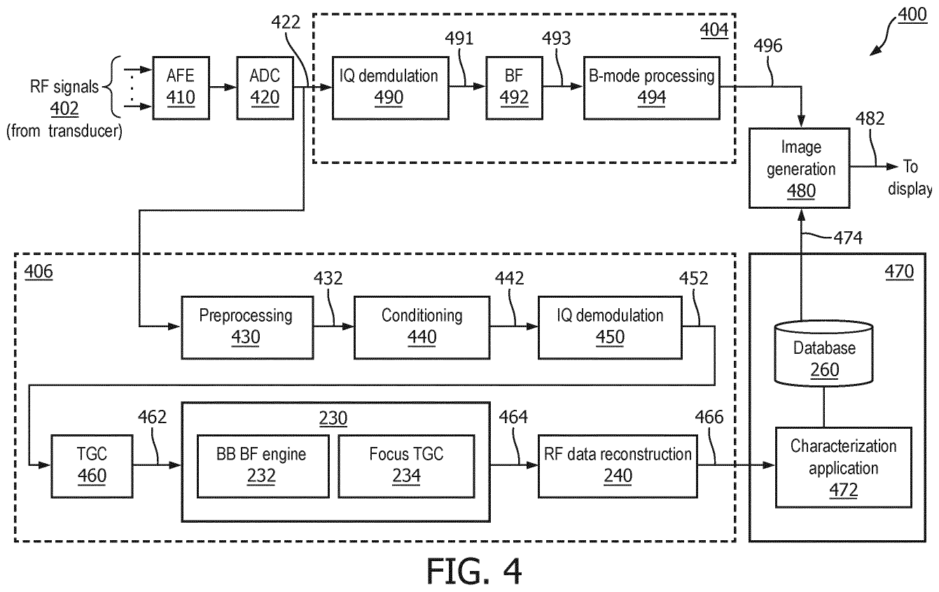

FIG. 4 is a schematic diagram illustrating an intraluminal ultrasound imaging system 400 that provides tissue characterization, according to aspects of the present disclosure. The system 400 is substantially similar to the systems 100 and 200. In an example, the system 400 may correspond to the system 200 with an updated receive signal path and configured to utilize the same database 260 generated by the system 200 for tissue characterization. For example, the system 400 may include one or more different hardware components, an additional hardware component, or a removal of a hardware component compared to the system 200. At a high-level, the system 400 is configured to match signal properties of ultrasound echo signals provided by the system 400 to signal properties of ultrasound echo signals provided by the system 200. The matching of the signal properties allows the system 400 to reuse the same validated database 260 to provide or reproduce histology information with high accuracy. The system 400 includes an analog frontend (AFE) 410, one or more ADCs 420, a B-mode processing signal path 404, and a VH processing signal path 406. The B-mode processing signal path 404 includes an IQ demodulation component 490, a BF component 492, and a B-mode processing component 494. The VH processing signal path 406 includes a preprocessing component 430, a conditioning component 440, an IQ demodulation component 450, a TGC component 460, the focusing engine 230 of FIG. 2, and the RF data reconstruction component 240 of FIG. 2.

The AFE 410 may be coupled to a transducer (e.g., the imaging component 110) and configured to receive ultrasound echoes from the transducer. The transducer may be used for imaging a vessel object (e.g., the vessel 120) and the ultrasound echoes are reflected by the vessel object. The ultrasound echoes may be in the form of per-channel raw analog RF signals 402, where each channel corresponds to an acoustic element in the transducer. The AFE 410 may include circuitry configured to provide gain controls and/or filtering. The gain controls can provide low-noise pre-amplification to the received echoes.

The one or more ADCs 420 are coupled to the AFEs 410. Each ADC 420 is configured to convert an analog RF channel signal 402 into a digital ultrasound echo channel signal. The one or more ADCs 420 output digital RF data 422 including a plurality of per-channel ultrasound echo data streams corresponding to the per-channel RF channel signals 402.

The IQ demodulation component 490, the BF component 492, and the B-mode processing component 494 in the B-mode processing signal path 404 and the preprocessing component 430, the conditioning component 440, the IQ demodulation component 450, and the TGC component 460 in the VH signal processing signal path can be implemented using any suitable combination of hardware and software components. In some embodiments, at least some of the components in the B-mode processing signal path and/or the VH signal processing path may be implemented on a field programmable gate array (FPGA) hardware. In some embodiments, at least some of the components in the B-mode processing signal path and/or the VH signal processing path may be implemented as software components executed by one or more processor cores, which may include general purpose processor core, a digital signal processing (DSP) core, a pipelined processor core, and/or a graphical processing unit (GPU).

In the B-mode processing path 404, the IQ demodulation component 490 is coupled to the one or more ADCs 420. The IQ demodulation component 490 is configured to receive the RF data 422 and down-convert the RF data 422 to BB IQ data 491. The BB conversion may include down-mixing, filtering, and decimating substantially similar to the operations of the IQ demodulation component 220. The BF component 492 is coupled to the IQ demodulation component 490 and configured to perform beamforming on the BB IQ data 491 to provide focused or beamformed data 493. The BF component 492 may include delay elements and summing elements configured to apply appropriate time-delays to the BB data and combine the time-delayed data to form beamformed data. The BF component 492 may be substantially similar to the BB BF engine 232. The B-mode processing component 494 is coupled to the BF component 492 and configured to perform B-mode processing to generate a B-mode image 496 (e.g., the B-mode image 152). The B-mode processing may include compounding, envelope detection, logarithmic compression, non-linear image filtering, and/or scan conversion. The B-mode image 496 may include a cross-section of the vessel object under the imaging represented by brightness levels or gray-scale. It should be noted that while FIG. 4 is illustrated with the BF component 492 following the IQ demodulation, in some other embodiments, beamforming can be performed on the RF data 422 before BB conversion.

In the VH processing path 406, the preprocessing component 430 is coupled to the one or more ADCs 420. The preprocessing component 430 is configured to receive the RF data 422 and preprocess the RF data 422 for tissue characterization and classification. The preprocessing may include truncating the RF data 422 to a field of view (FOV) (e.g., of about 10 millimeter) matching a FOV used in the system 200 for generating the database 260.

The conditioning component 440 is coupled to the preprocessing component 430 and configured to condition the preprocessed RF data 432 to match the RF signal properties (e.g., frequency spectrum and signal amplitudes) of the RF data 212 in the system 200. While the system 400 may use the same ultrasound transducer (e.g., the imaging component 110) as the system 200, the hardware components (e.g., filters, circuitries, AFE 410 and the ADCs 420) along the receive signal path of the system 400 can be different from the hardware components (e.g., filters, circuitries, and the frontend processing component 210) along the receive signal path of the system 200. As such, the RF data 432 generated by the system 400 may have different signal properties than the RF data 212 generated by the system 200.

In an embodiment, the conditioning component 440 may apply an FIR bandpass filter to the RF data 432. The FIR bandpass filter may be configured to provide a frequency response matching the frequency spectrum of the RF data 212 since tissue characterization is based on frequency properties (e.g., power spectral properties) of ultrasound echo responses.

The IQ demodulation component 450 is coupled to the conditioning component 440 and configured to down-convert the conditioned RF data 442 to BB IQ data 452. The IQ demodulation component 450 can be substantially similar to the IQ demodulation component 490. The BB conversion may include down-conversion, filtering, and/or decimation substantially similar to the operations performed by the IQ demodulation component 220 in the system 200. However, in some embodiments, the IQ demodulation component 450 and the IQ demodulation component 220 may not include identical implementations.

The TGC component 460 is coupled to the IQ demodulation component 450 and configured to apply time-gain compensation to the BB IQ data 452. In some embodiments, the TGC component 460 may provide time-gain compensated BB data 462 with signal properties matching signal properties of the BB IQ data 222 of the system 200.

In an embodiment, the system 400 can reuse the focusing engine 230 from the system 200 (in FIG. 2) to generate focused BB data 464. The system 400 can further reuse the RF data reconstruction component 240 from the system 200 (in FIG. 2) to generate RF data 466 for tissue characterization. The RF data 466 in the system 400 may be include signal properties substantially matching the signal properties of the RF data 242 in the system 200.

The system 400 further includes a tissue characterization component 470. The tissue characterization component 470 may be substantially similar to the tissue characterization component 140 of FIG. 1. The tissue characterization component 470 includes the database 260 (e.g., generated using the system 200) and a characterization application 472. The characterization application 472 is configured to receive the RF data 466 and determine tissue composition information in the RF data 466 using the database 260. The characterization application 472 can use similar mechanisms as in the tissue characterization data generation component 250 and the scheme 300 discussed above with respect to FIGS. 2 and 3, respectively, to determine power spectral properties of the RF data 466.

For example, the characterization application 472 transforms the RF data 466 into the frequency domain. The characterization application 472 determines power spectral parameters similar to the y-intercept parameter 301, the maximum power parameter 302, the maximum power frequency location 305, the mid-band parameter 303, the minimum power parameter 304, the minimum power frequency location 306, the slope of the linear regression line 328, and/or the integrated backscatter data discussed above with respect to FIG. 3. The characterization application 472 can determine power spectral parameters for each scan line in an ROI (e.g., the ROI 310) portion of the RF data 466. The characterization application 472 can further determine power spectral parameters for each segment of a scan line in the ROI. The characterization application 472 correlates the determined power spectral parameters to the parameters stored in the database 260 to identify tissue types associated with the RF data 466.

In an embodiment, the database 260 may be stored as a classification tree (e.g., the tree 340) as shown in FIG. 3. In such an embodiment, the characterization application 472 inputs the determined power spectral parameters to the classification tree and the classification tree can be traversed in accordance with the branching conditions and lead to a leaf node (e.g., the leaf nodes 368) that identifies the type (e.g., calcified, fibrous, fibro-lipidic, and calcified necrosis) of tissue matching the inputted power spectral parameters.

The tissue characterization component 470 outputs tissue characterization data 474 that can be used to create a VH image (e.g., the VH image 154) for display. The tissue characterization data 474 can include tissue composition information for a portion of the received RF signals 402 corresponding to the FOV determined by the preprocessing component 430. The system 400 further includes an image generation component 480 coupled to the B-mode processing component 494 and the tissue characterization component 470. The image generation component 480 is configured to create a VH image from the tissue characterization data 474 and combine the VH image and the B-mode image 496 to output a combined image 482 for display.

In an embodiment, the image generation component 480 is configured to identify a region in the B-mode image 496 matching the VH image. For example, the image generation component 480 may identifying landmarks in both the B-mode image 496 and the VH image. The landmarks may include, but not limited to, side branch vessels, identifiable plaque or calcium deposits and all other vascular tissue landmarks generally known to those skilled in the art. The image generation component 480 may morph and/or warp the VH image to substantially fit the contour of the B-mode image 496. The morphing and/or warping may include applying a morphometric algorithm and/or a thin plate spline (TPS) deformation algorithm to substantially align the non-landmark portions of the B-mode image 496 and the VH image.

In an embodiment, the image generation component 480 is configured to create the combined image 482 by overlaying the aligned, morphed, warped VH image (e.g., the VH image 154) on top of B-mode image 496.

In an embodiment, the tissue characterization component 470 and/or the image generation component 480 may be implemented by a host computing machine separate from the processing system (e.g., the processing system 106 and/or the PIM 104) where the B-mode processing signal path 404 and the VH processing signal path 406 reside. In some embodiments, at least a portion of the tissue characterization component 470 and/or the image generation component 480 may be implemented on the same system where the B-mode processing signal path 404 and the VH processing signal path 406 reside.

In some embodiments, the system 400 can further include components configured to provide analog spectrum compensation, analog spectrum gain, and TGC such that the signal properties of the system 400 are better matched to the signal properties of the system 200. The better matching can provide a more accurate tissue characterization result.

The system 200 can be further configured to provide B-mode images (e.g., the B-mode images 152 and 496) and VH images (e.g., the VH image 154) after generating the tissue characterization database 260 similar to the system 400. For example, the system 200 can include a B-mode processing signal path substantially similar to the B-mode processing signal path 404 of the system 400. The system 200 can include a tissue characterization component and an image generation component substantially similar to the tissue characterization component 470 and the image generation component 480 of FIG. 4, respectively.

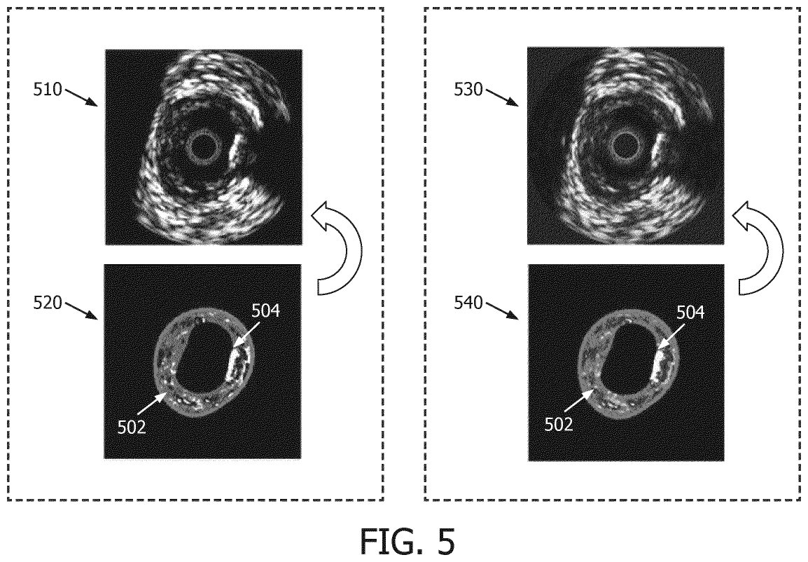

FIG. 5 illustrates example B-mode images and VH images generated from intraluminal ultrasound imaging systems, according to aspects of the present disclosure. The left-side of FIG. 5 shows a B-mode image 510 and a corresponding VH image 520 generated from the system 200. The B-mode image 510 and the VH image 520 are generated from ultrasound echo data acquired using the system 200 when imaging a vessel object (e.g., the vessel 120). The right-side of FIG. 5 shows a B-mode image 530 and a corresponding VH image 540 generated from ultrasound echo data acquired using the system 400 when imaging the same vessel object. Both VH images 520 and 540 are generated based on the same database 260 generated by the system 200. Each of the B-mode images 510 and 530 illustrate a cross-section of the vessel object in gray-scale. Each of the VH images 520 and 540 illustrates a tissue composition map for an ROI in the corresponding cross-section. While the VH images 520 and 540 are shown as gray-scale in FIG. 5, the VH images 520 and 540 can be color-coded to illustrate the different tissue types. For example, tissues with a necrotic core, tissues with dense calcium, fibrous tissues, and fibro-fatty tissues can be shown as red, white, dark green, and light green, respectively.

As can be observed, the B-mode image 510 generated from the system 200 and the B-mode image 530 generated from the system 400 include substantially similar speckle patterns. In addition, the VH image 520 generated from the system 200 and the VH image 540 generated from the system 400 include substantially similar tissue composition mapping including fibrous plaque 502 with a calcified region 504. As described above, the system 200 and the system 400 may include different signal properties in the receive signal path, for example, due to different frontend hardware and/or other implementations. However, the conditioning component 440 in the system 400 matches the differences in signal properties between the systems 200 and 400. Accordingly, the system 400 can use the tissue characterization database 260 generated from the system 200 to provide substantially accurate tissue characterization information instead of creating a new tissue characterization database 260 using the system 400.

It should be noted that the B-mode image 510 and the VH image 520 are shown as separate images for simplicity of illustration. However, the VH image 520 can be displayed as an overlay on top of the B-mode image 510. Similarly, the VH image 540 can be displayed as an overlay on top of the B-mode image 530.

FIG. 6 is a schematic diagram of a processor circuit 600, according to embodiments of the present disclosure. The processor circuit 600 may be implemented in the PIM 104 and/or the processing system 106 of FIG. 1. As shown, the processor circuit 600 may include a processor 660, a memory 664, and a communication module 668. These elements may be in direct or indirect communication with each other, for example via one or more buses.

The processor 660 may include a CPU, a DSP, an application-specific integrated circuit (ASIC), a controller, an FPGA, another hardware device, a firmware device, or any combination thereof configured to perform the operations described herein, for example, aspects of FIGS. 1-4 and 7. The processor 660 may also be implemented as a combination of computing devices, e.g., a combination of a DSP and a microprocessor, a plurality of microprocessors, one or more microprocessors in conjunction with a DSP core, or any other such configuration.

The memory 664 may include a cache memory (e.g., a cache memory of the processor 660), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 664 includes a non-transitory computer-readable medium. The memory 664 may store instructions 666. The instructions 666 may include instructions that, when executed by the processor 660, cause the processor 660 to perform the operations described herein, for example, aspects of FIGS. 1-4 and 7 and with reference to the PIM 104 and/or the processing system 106 (FIG. 1). Instructions 666 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 668 can include any electronic circuitry and/or logic circuitry to facilitate direct or indirect communication of data between the processor circuit 600, the imaging device 102, and/or the display 108. In that regard, the communication module 668 can be an input/output (I/O) device. In some instances, the communication module 668 facilitates direct or indirect communication between various elements of the processor circuit 600 and/or the PIM 104 (FIG. 1) and/or the processing system 106 (FIG. 1).

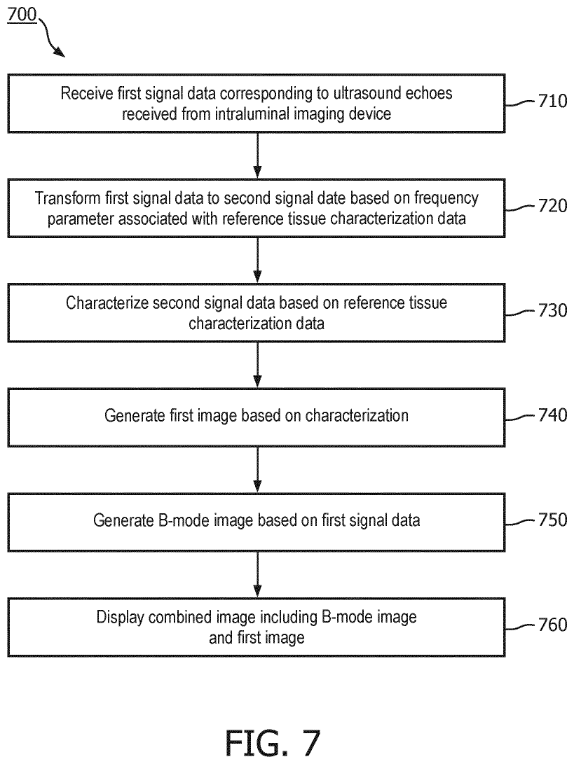

FIG. 7 is a flow diagram of a tissue characterization method 700, according to aspects of the disclosure. Steps of the method 700 can be executed by the systems 100, 200, and/or 400, for example, by a processor circuit such as the processor circuit 600, and/or other suitable component such as the imaging component 110, the PIM 104, and/or the processing system 106. the processor circuit 600, and/or any suitable combination of components in the system 200 and/or the system 400. The method 700 may employ similar mechanisms as in the systems 100, 200, and 400 described with respect to FIGS. 1, 2, and 4, respectively, and the scheme 300 described with respect to FIG. 3. As illustrated, the method 700 includes a number of enumerated steps, but embodiments of the method 700 may include additional steps before, after, and in between the enumerated steps. In some embodiments, one or more of the enumerated steps may be omitted or performed in a different order.

At step 710, the method 700 includes receiving, at a processor circuit in communication with an intraluminal imaging device including an ultrasound transducer array, first signal data corresponding to ultrasound echoes associated with a body lumen. In some examples, the processor circuit may be similar to the processor circuit 600. In some examples, the processor circuit may be located at the PIM 104. In some examples, the processor circuit may be located at the processing system 106. In some examples, the processor circuit may include circuit components distributed across the PIM 104 and the processing system 106. The intraluminal imaging device may be similar to the imaging device 102. The ultrasound transducer array may be similar to the imaging component 110. The first signal data may be similar to the RF data 202, 242, 312, 402, and/or 466. The body lumen may be similar to the vessel 120. In an embodiment, the first signal data includes RF backscatter data corresponding to the ultrasound echoes.

At step 720, the method 700 includes transforming the first signal data to second signal data based on a first parameter associated with a reference tissue characterization data. The reference tissue characterization data may be similar to the database 260 shown in FIGS. 2, 3, and 4. In an embodiment, the transformation may include performing substantially similar operations (e.g., conditioning, focusing, and/or RF data reconstruction) as components along the VH processing signal path 406.

In an embodiment, the first parameter includes a frequency parameter associated with the reference tissue characterization data. The frequency parameter may be associated with an ultrasound imaging system (e.g., the system 200) used for generating the reference tissue characterization data. For example, the frequency parameter may be associated with one or more of the y-intercept parameter 301, the maximum power parameter 302, the maximum power frequency location 305, the mid-band parameter 303, the minimum power parameter 304, the minimum power frequency location, the slope of the linear regression line 328, and/or the integrated backscatter data discussed above with respect to FIG. 3. In some embodiments, the first parameter may be a frequency parameter associated with ultrasound echo signals generated from a particular system (e.g., the system 200) and the reference tissue characterization data may be generated using the system.

In some embodiments, the first parameter may be determined by comparing signal characteristics of ultrasound echoes generated by a first ultrasound imaging system (e.g., the systems 100 and/or 200) and a second ultrasound imaging system (e.g., the systems 100 and/or 400), where the reference tissue characterization data is generated from the first system. In some examples, the first system may be a manufacturer system used for generating the reference tissue characterization data and the second system may be a user system that is implemented in a clinical setting. In some examples, the first system may be a previous generation system and the second system may be a current generation system. In some examples, the first system may be a current generation system and the second system may be a future generation system. In general, the first system and the second system are different and may have different ultrasound data acquisition signal paths. In some embodiments, the first parameter may be determined by comparing signal characteristics of ultrasound echoes generated by an ultrasound imaging system (e.g., the system 100) before and after an upgrade, where the reference tissue characterization data is generated from the system before the upgrade.

In an embodiment, the transforming includes applying a filter to the first signal data. The filter may be determined based on the first parameter, for example, using the conditioning component. In an embodiment, the transforming includes applying the filter to a portion of the first signal data based on a FOV parameter associated with the reference tissue characterization data. For example, the first signal data is truncated to a certain FOV using the preprocessing component 430. In some other embodiments, the filter coefficients can be determined based on a comparison of signal characteristics of ultrasound data acquired from a first ultrasound imaging system (e.g., the system 200) and a second ultrasound imaging system (e.g., the system 400), where the first system is used for generating the reference tissue characterization data and the second system is in use, for example, during an ultrasound examination.

At step 730, the method 700 includes characterizing the second signal data based on the reference tissue characterization data, for example, using the tissue characterization component 470.

At step 740, the method 700 includes generating a first image (e.g., the VH images 154, 520 and 540) of the body lumen based on the characterization.

At step 750, the method 700 includes generating a B-mode image (e.g., the B-mode images 150, 496, 510, and 530) of the body lumen based on the first signal data.

At step 760, the method 700 includes outputting, to a display (e.g., the display 108) in communication with the processor circuit, a combined image including the first image and the B-mode image.

In an embodiment, the method 700 further includes converting the second signal data to BB signal data (e.g., the BB IQ data 452), for example, using the IQ demodulation component 450. The method 700 further includes performing beamforming on the BB signal data to produce focused data (e.g., the focused data 464), for example, using the focusing engine 230. The method 700 further includes converting the focused data to RF signal data (e.g., the focused RF data 466), for example, using the RF data reconstruction component 240. The characterizing includes characterizing the RF signal data based on the reference tissue characterization data.

In an embodiment, the method 700 further includes adjusting at least one of a time parameter or a gain parameter of the first signal data based on a second parameter associated with the reference tissue characterization data, for example, using a TGC component similar to the TGC components 460 and/or 234.

In an embodiment, wherein the first image includes a tissue composition map of the body lumen. The outputting includes outputting the first image as an overlay on top of the B-mode image.

Aspects of the present disclosure can provide several benefits. For example, the inclusion of a conditioning component (e.g., the conditioning component 440) in an upgraded intraluminal system (e.g., the system 400) to match spectral properties of raw RF backscatter data acquired from the system before the upgrade allows the reuse of a tissue characterization database (e.g., the database 260) generated and validated before the upgrade. The reuse of a well-validated tissue characterization database instead of creating a new tissue characterization database after each system upgrade can save time and cost. While the disclosed embodiments are described in the context of reusing a well-validated tissue characterization database after a system upgrade, similar conditioning mechanisms can be applied to any intraluminal imaging system to match the RF backscatter signal characteristics of the intraluminal imaging system used for generating the tissue characterization database.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ultrasound imaging system, comprising:
an intraluminal imaging device comprising a flexible elongate member configured to be positioned within a body lumen of a patient and an ultrasound transducer array coupled to the flexible elongate member, the ultrasound transducer array configured to transmit ultrasound energy into the body lumen and to receive ultrasound echoes associated with the body lumen; and
a processor circuit in communication with the intraluminal imaging device and configured to:
receive, from the ultrasound transducer array, first signal data corresponding to the received ultrasound echoes;
transform the first signal data to second signal data based on a first parameter associated with a reference tissue characterization data;
characterize the second signal data based on the reference tissue characterization data;
generate a first image of the body lumen based on the characterization; and
output, to a display in communication with the processor circuit, the first image.

2. The system of claim 1, wherein the first signal data includes radio frequency (RF) backscatter data corresponding to the received ultrasound echoes.

3. The system of claim 1, wherein the first parameter includes a frequency parameter associated with the reference tissue characterization data.

4. The system of claim 1, wherein the first parameter includes a power spectral parameter associated with the reference tissue characterization data.

5. The system of claim 1, wherein the processor circuit configured to transform the first signal data to the second signal data includes:
applying a filter to the first signal data, the filter determined based on the first parameter.

6. The system of claim 5, wherein the processor circuit configured to transform the first signal data to the second signal data includes:
applying the filter to a portion of the first signal data based on a field of view (FOV) parameter associated with the reference tissue characterization data.

7. The system of claim 1, wherein the processor circuit is further configured to:
convert the second signal data to baseband signal data;
perform beamforming on the baseband signal data to produce focused data; and
convert the focused data to radio frequency (RF) signal data, and
wherein the processor circuit configured to characterize the second signal data is further configured to characterize the RF signal data based on the reference tissue characterization data.

8. The system of claim 1, wherein the processor circuit is further configured to:
adjust at least one of a time parameter or a gain parameter of the first signal data based on a second parameter associated with the reference tissue characterization data.

9. The system of claim 1, wherein the processor circuit is further configured to:
generate a brightness-mode (B-mode) image of the body lumen based on the first signal data; and
output, to the display, a combined image including the B-mode image and the first image, wherein the first image includes a tissue composition map of the body lumen.

10. The system of claim 1, wherein the reference tissue characterization data is associated with at least one of a calcified tissue type, a fibrous tissue type, a fibro-lipidic tissue type, or a calcified necrosis tissue type.

11. The system of claim 1, wherein the processor circuit includes a field programmable gate array (FPGA) configured to transform the first signal data to the second signal data based on the first parameter associated with the reference tissue characterization data.

12. The system of claim 1, further comprising:
a memory configured to store program code for transforming the first signal data to the second signal data based on the first parameter associated with the reference tissue characterization data,
wherein the processor circuit includes a processor in communication with the memory, and
wherein the processor circuit configured to transform the first signal data to the second signal data includes executing the program code by the processor.

13. A method of ultrasound imaging, comprising:
receiving, at a processor circuit in communication with an intraluminal imaging device including an ultrasound transducer array, first signal data corresponding to ultrasound echoes associated with a body lumen;
transforming the first signal data to second signal data based on a first parameter associated with a reference tissue characterization data;
characterizing the second signal data based on the reference tissue characterization data;
generating a first image of the body lumen based on the characterization; and
outputting, to a display in communication with the processor circuit, the first image.

14. The method of claim 13, wherein the first signal data includes radio frequency (RF) backscatter data corresponding to the ultrasound echoes.

15. The method of claim 13, wherein the first parameter includes a frequency parameter associated with the reference tissue characterization data.

16. The method of claim 13, wherein the transforming includes:
applying a filter to the first signal data, the filter determined based on the first parameter.

17. The method of claim 16, wherein the transforming includes:
applying the filter to a portion of the first signal data based on a field of view (FOV) parameter associated with the reference tissue characterization data.

18. The method of claim 13, further comprising:
converting the second signal data to baseband signal data;
performing beamforming on the baseband signal data to produce focused data; and
converting the focused data to radio frequency (RF) signal data, and
wherein the characterizing includes:
characterizing the RF signal data based on the reference tissue characterization data.

19. The method of claim 13, further comprising:
adjusting at least one of a time parameter or a gain parameter of the first signal data based on a second parameter associated with the reference tissue characterization data.

20. The method of claim 13, further comprising:
generating a B-mode image of the body lumen based on the first signal data,
wherein the outputting includes:
outputting, to the display, a combined image including the B-mode image and the first image, wherein the first image includes a tissue composition map of the body lumen.
